(19) United States
(12) Patent Application Publication
(10) Pub. No.: US 2005/0065585 A1
(43) Pub. Date: Mar. 24, 2005

(54) Heat generating therapeutic device for ears

(76) Inventor: Javier Salas, Cancun (MX)

Correspondence Address: B. Craig Killough, 865 Island Park Drive, Charleston, SC, 29492, US

(21) Appl. No.: 10/701,768

(22) Filed: Nov. 5, 2003

Related U.S. Application Data
(60) Provisional application No. 60/424,366, filed on Nov. 5, 2002.

(51) Int. Cl. A61F 7/00

(52) U.S. Cl. 607/109; 607/112

(57) ABSTRACT

A device may be worn on the head and surround the outer ear. The device comprises one or more materials that exotherm upon activation, so that the device generates heat. The heat is contained within the ear cap or covering for the outer ear, and the heat is communicated with the ear canal. The heat reduces pain associated with infection or pressure changes, and assists in equalizing pressure within the ear.

The Applicant claims priority from U.S. Provisional Application Ser. No. 60/424366 filed Nov. 5, 2002.

### FIELD OF THE INVENTION

This invention relates to therapeutic devices generally, as more specifically directed to a heat generating device that is worn over the ears.

### BACKGROUND OF INVENTION

Infections of the ear and unequal pressure in the ear can result in significant pain. For example, unequal pressure in the ear caused by altitude changes while traveling in airplanes can result in significant pain, particularly in children. Children are also particularly susceptible to infections which cause significant ear pain. A device that is transportable and can be easily applied to assist in relieving pain in the ear caused by pressure changes and/or by infections is needed.

### SUMMARY OF THE INVENTION

The present invention is a device that may be worn on the head and surround the outer ear. The device comprises one or more materials that exotherm upon activation, so that the device generates heat. The heat is contained within the ear cap or covering for the outer ear, and the heat is communicated with the ear canal. The heat reduces pain associated with infection or pressure changes, and assists in equalizing pressure within the ear. Pain that is the result of acute external otitis, temporomandibular joint dysfunction, acute otitis media, ear barotrauma and otalgia due to barometric pressure changes may be treated with the invention.

### DESCRIPTION OF DRAWINGS

FIG. 1 is a front elevation of an embodiment of the present invention.

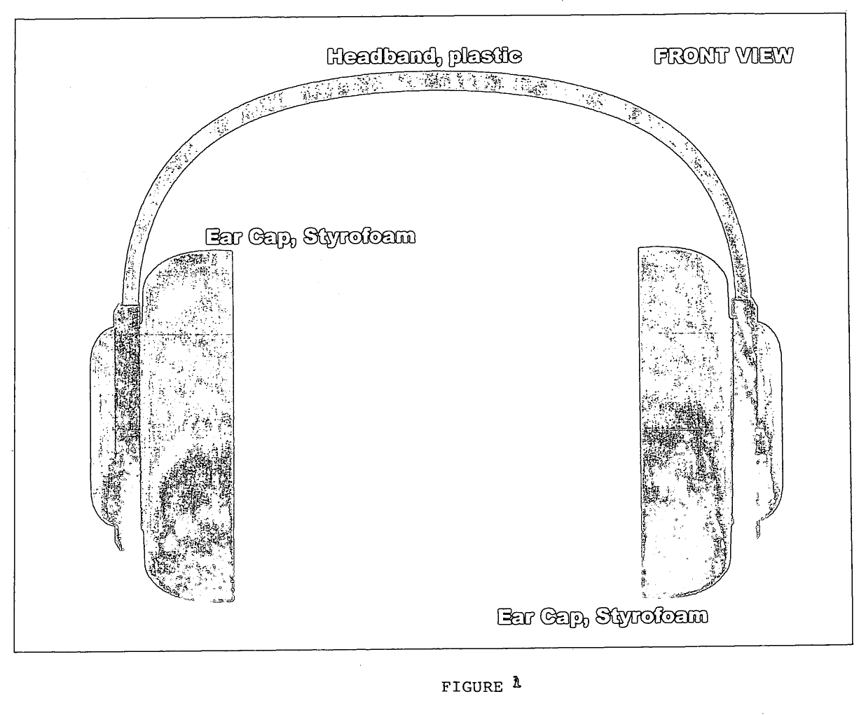

FIG. 2 is a top plan view of an embodiment of the present invention.

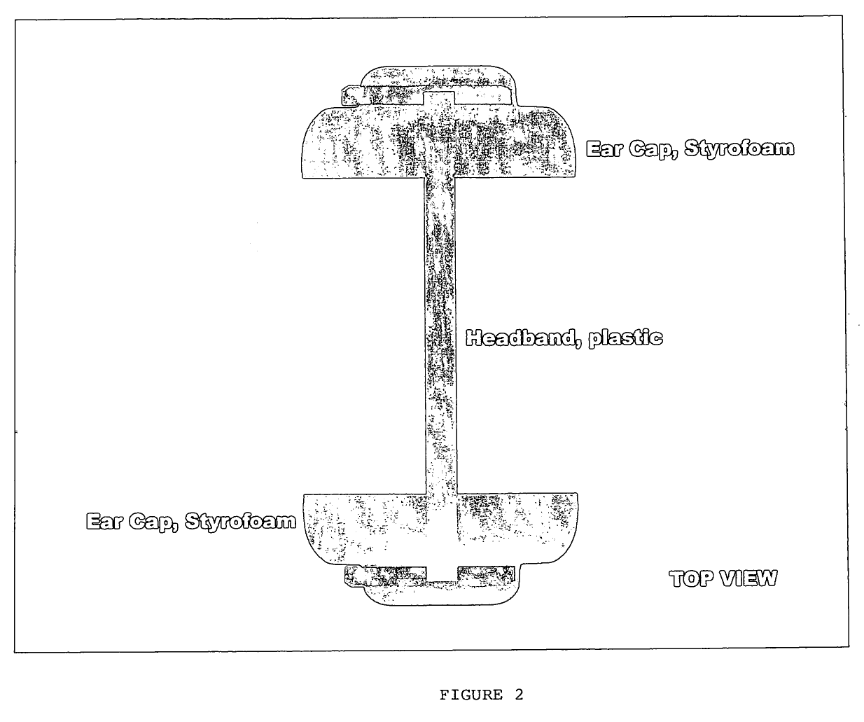

FIG. 3 is a side view of an embodiment of the present invention.

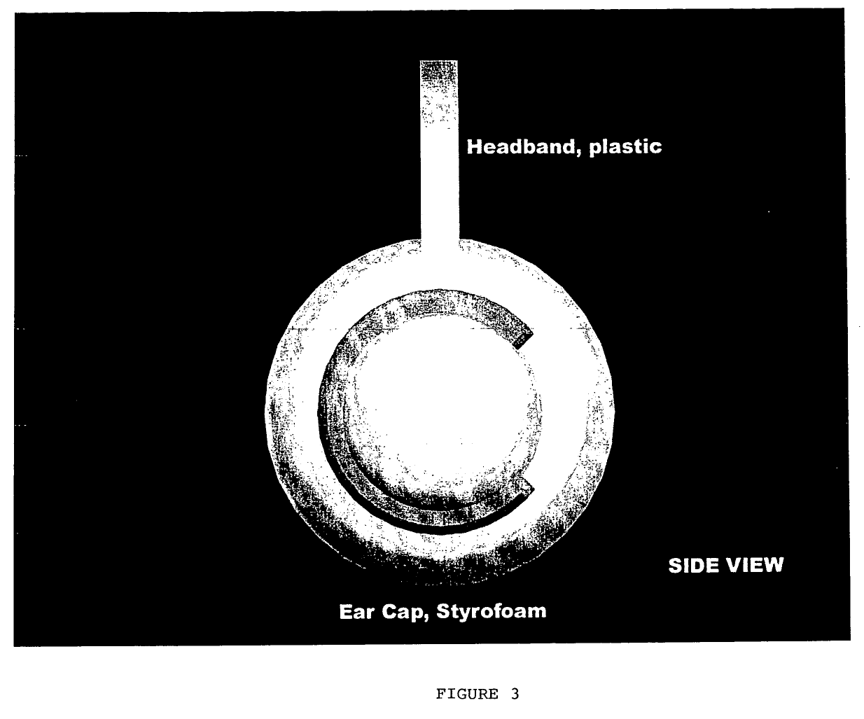

FIG. 4 is perspective view of an embodiment of the present invention.

FIG. 5 is an additional perspective view of an embodiment of the present invention.

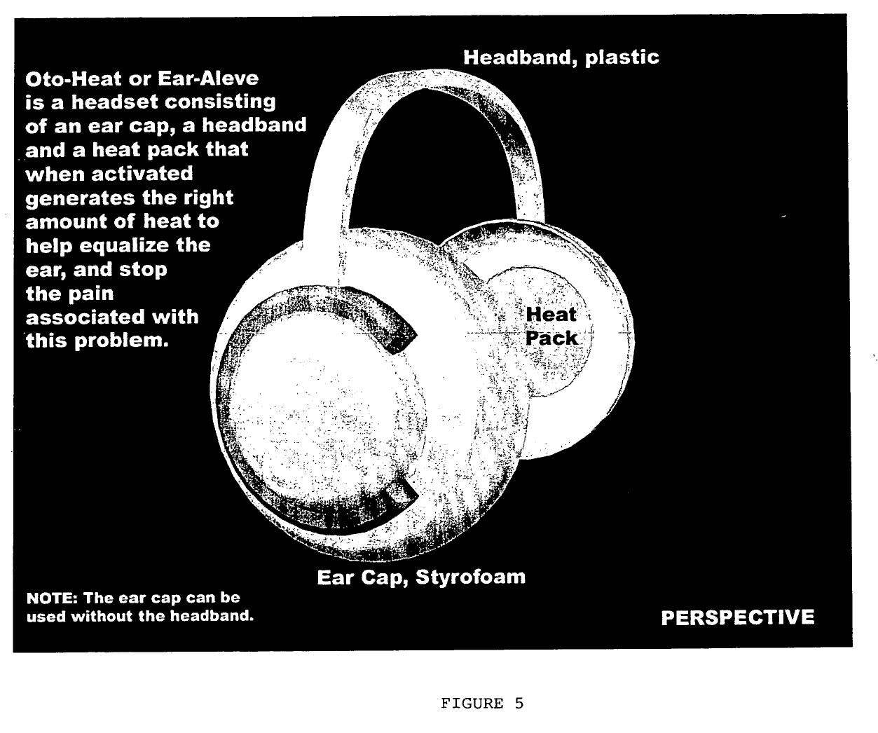

### DETAILED DESCRIPTION OF THE PREFERRED EMBODIMENT

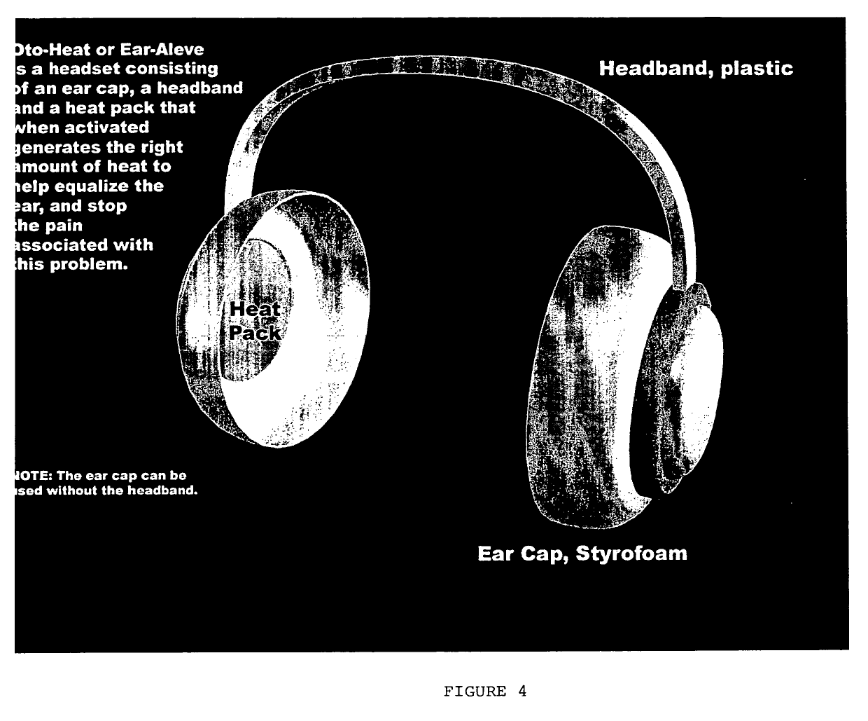

Referring now to the drawing figures, FIG. 4 shows a preferred embodiment of the device. The device has a left ear cap, a right ear cap and a headband that joins the left ear cap to the right ear cap. At least one of the right ear cap or left ear cap comprises a heat pack. The heat pack comprises one or more materials that exotherm and generate heat upon activation, such as by the mixing of materials.

In one embodiment of the invention, a heat pack is present within in the ear cap. The heat pack comprises a container that may be deformed by manual pressure. The container is not susceptible to puncture, rupture or breakage from manual pressure, or from “normal” impact, such as being dropped, or the like. Within the container is a thin membrane that separates a first material from a second material. The first material and second material materials are contained within the container, and are separated by the thin membrane, so that they do not mix of react with each other until the device is ready for use. The membrane is therefore impermeable by the first material and second material.

The membrane that is present within the container is subject to rupture or bursting on manual pressure that is applied to the container and/or the materials contained therein. When manual pressure is applied to the container membrane, such as by pressing on a portion of the ear cap that is adjacent to the larger membrane, the pressure is created inside the container and against the materials causes the membrane to rupture, so that the previously separated materials now encounter each other, and are mixed. As the materials mix, they exotherm to produce usable heat.

The structure of the ear cap directs heat toward the ear and ear canal. it is preferred that the exterior of the ear cap have a high insulation co-efficient. The ear cap is structured so that the container is adjacent to the ear, and is not insulated by a portion of the ear cap. In this way, the container is substantially next to the outer ear. The heat formed by the heat pack is contained within the interior of the ear cap, and is transmitted to the ear.

The exothermic materials may be known materials which will generate heat upon activation, such as two or more materials that exotherm upon contact with each other, or when reacted with each other. The materials are preferred to be non-toxic, and not likely to cause chemical burns to the skin in the event that the container is ruptured. Further, it is preferred that the materials not be particularly harmful to textiles, furniture and other objects that may be encountered in the environment in which the device is used. An example of materials which will fulfill the objects of the present invention are calcium chloride and water. When calcium chloride and water are mixed,.they will exotherm so as to reduce the required heat. For example, the heat pack may comprise 8 grams of calcium chloride and 5 cc of water.

The device may comprise one or two ear caps and one or two heat packs. In the preferred embodiment, as shown in the drawings, the device comprises two ear caps that are connected by a headband, so that the device has the appearance of headphones. The ear caps are structured so that the internal edge of each ear cap rests substantially against the head of the wearer, and heat is retained within the internal portion space of the ear cap created when the ear cap is pressed against the ear. The ear cap may be formed of plastic or other materials, and may comprise a material that deforms to form the shape of the head at the edge of the ear cap that joins the head.

In use, the ear cap is deformed by manual pressure at a portion of the ear cap that adjacent to the heat pack. Sufficient pressure is applied to break the thin membrane within the heat pack so that heat is generated. The device is then immediately placed over the head of the wearer, with the ear cap over the ear. The heat pack warms the interior space of the ear cap and warms the outer ear, and heat is received into the ear canal. This heat assists in reducing ear pain in adults and children, and will also assist in equalizing pressure within the ear. In some cases, the heat will assist in removing blockages in the ear and Eustachian tube to assist in relieving pressure, such as that cause by altitude changes while flying.

1. A heat generating therapeutic device for ears, comprising:
a) at least one ear cap that is adapted to rest against substantially against a head of a user, and to cover an outer ear of a user; and
b) a heat pack that is contained within said ear cap, wherein said hear pack comprises a first material and a second material, wherein said first material and said second material will exotherm when combined, wherein said first material and said material are separated within said heat pack, and wherein said first material and said second material are combined by the application of pressure to said heat pack.

2. A heat generating therapeutic device for ears as described in claim 1, further comprising means for holding said at least one ear cap so that said at least one ear cap rests substantially against the head of the user.

3. A heat generating therapeutic device for ears as described in claim 1, wherein said first material and said second material are separated within said heat pack by a membrane.

4. A heat generating therapeutic device for ears as described in claim 3, wherein said membrane is adapted to rupture upon the application of manual pressure to said membrane.

5. A heat generating therapeutic device for ears as described in claim 1, wherein said at least one ear cap comprises a first ear cap and a second ear cap, wherein said first ear cap is positioned opposite said second ear cap, and wherein said first ear cap is adapted to cover a right ear of a user and said second ear cap is adapted to cover a left ear of a user.

6. A heat generating therapeutic device for ears as described in claim 5, wherein said first ear cap and said second ear cap are connected by a head band that is adapted to rest over the head of a user.
